(19) United States
(12) Patent Application Publication
(10) Pub. No.: US 2006/0078630 A1
(43) Pub. Date: Apr. 13, 2006

(54) Method for the production of flavonoid-containing compositions and use thereof

(76) Inventors: Christoph Mathis Schempp, Freiburg (DE); Axel Wahling, Magdeburg (DE); Elfriede Lange, Magdeburg (DE)

Correspondence Address: SMITH PATENT CONSULTING CONSULTING, LLC, P.O. BOX 2726, ALEXANDRIA, VA, 22301, US

(21) Appl. No.: 10/545,932

(22) PCT Filed: Feb. 19, 2004
(86) PCT No.: PCT/EP04/01598

§ 371 Date: Aug. 18, 2005

(30) Foreign Application Priority Data
Feb. 26, 2003 (DE) 10308162.3

(51) Int. Cl. A61K 36/18; A61K 31/7048; A61K 31/353

(52) U.S. Cl. 424/725; 514/27; 514/456

(57) ABSTRACT

The present invention relates to a method for the production of plant extracts having a standardized flavonoid content, in particular having a standardized luteolin content. Plant material of Reseda plants which has been pre-extracted with water is extracted with a solvent and then concentrated. The compositions thus obtained are suitable as pharmaceutical or cosmetic products and as colorants. The invention furthermore relates to the use of Reseda extracts for treating skin diseases.

The present invention relates to a method for the production of flavonoid-containing compositions from plants of the Resedaceae family. The compositions, and intermediates obtained in the production, are advantageously used for dyeing natural textiles and leather and for the production of coloured pigments. The compositions, and intermediates obtained in the production, can also be used in cosmetics and medicaments.

The Resedaceae family comprises, inter alia, the plant species Reseda alba L., Reseda glauca L., Reseda lutea L., Reseda odorata L., Reseda phyteuma L. and Reseda L. For centuries, Reseda luteola, also referred to as dyer's rocket, has been used for dyeing natural textiles in yellow and green shades, also in combination with other dyer's plants. Up to the 20th century, the plant was used for dyeing silk. H. Schweppe (Handbuch der Naturfarbstoffe [Handbook of Natural Dyes], ecomed Verlag, 1993) describes the use, analysis and historical development of the use of this plant in textile dyeing. Later, the dye was completely replaced by synthetic dyes.

Reseda luteola can be easily cultivated, and the plant prefers lime-containing soils. Recent research work by Vetter and Biertümpfel (conference volumes 1997 and 1999 of the Forum on Dye Plants; FNR Gülzow) on the cultivation of Reseda luteola in Germany describes the agricultural potential as a renewable raw material.

In addition to the main components sugars, proteins and fibres, Reseda luteola contains secondary plant ingredients, flavonoids being of interest. Inter alia, the flavonoids luteolin, apigenin, luteolin-7-glucoside and luteolin-3,7-glucoside (H. Schweppe, Handbuch der Naturfarbstoffe [Handbook of Natural Dyes], ecomed Verlag, 1993; L. Adam, conference volume of the Forum of Dyer's Plants 1999, K. Loest, concluding report on research project FNR 97NR147-F, 2001) have been detected.

The luteolin contained in Reseda luteola constitutes the colouring principle. It belongs to the group consisting of flavonoids, i.e. plant ingredients which are widespread in the plant kingdom. Flavonoids have a multiplicity of biological activities. For example, cardioprotective activity was reported for luteolin, in that it prevents the oxidation of lipids and inhibits cholesterol synthesis (Cook et al. (1996) Nutritional Biochemistry 7, 66; Pietta (2000), J. Nat. Prod. 63, 1035). Furthermore, an analgesic and anti-inflammatory activity of luteolin is known (Toreda et al. (1994) Z. Naturforschung, C49, 35). Moreover, mast cell-stabilizing effects of flavonoids, such as luteolin and quercetin, were described (Kimata et al. (2000) Chemical and Experimental Allergy, 30, 501). In vitro, numerous cytostatic effects of luteolin and other flavonoids were described.

Furthermore, antibacterial activity of luteolin (Pettit et al., (1996) J. Ethnopharmacol. 53, 57) and antioxidant activity of flavonoids (Pietta (2000) J. Nat. Prod. 63, 1035) are known. WO 00/26206 A1 describes a method for producing luteolin and derivatives thereof. In the area of dermatological cosmetics, various antioxidants, such as vitamin C and vitamin E, are used. Moreover, retinoids are used for retarding light-related ageing of skin. Plant extracts from Potentilla erecta improve where possible the anchoring of the epidermis in the basal membrane and can lead to tauter skin (WO 98.19664).

Dyeing with plant dyes is currently limited to arts and crafts. Aqueous decoctions of the plant parts are used for dyeing. This procedure has two decisive disadvantages. The dyes of the plants are dissolved only to an insufficient extent since particularly the aglycones luteolin and apigenin are sparingly soluble. A further disadvantage is the use of the plant parts, which is not possible in industrially operating finishing units on high-speed machines. The synthetic production of luteolin is complicated and expensive. FR 2 632 523 A1 discloses the extraction of various plants with ethanolic solutions, water or hexane.

It is an object of the present invention to provide a flavonoid-containing composition having a standardized flavonoid content, in particular luteolin content.

Surprisingly, it was found that compositions having a high luteolin content are obtained if the plant material is pre-extracted with an aqueous solution before an extraction of Reseda plants with a solvent. The invention therefore relates to a method for the production of a flavonoid-containing composition, which comprises a) extracting material of at least one plant of the Resedaceae family, which has been pre-extracted with an aqueous solution, with a first solvent; and b) removing at least a part of the first solvent from the extract obtained in step a).

The plant material used may originate from any desired plant of the Resedaceae family. Examples are the plant species Reseda alba L., Reseda glauca L., Reseda lutea L., Reseda odorata L., Reseda phyteuma L. and Reseda luteola L. Reseda luteola L. is preferred. Mixtures of materials of a plurality of plant species may also be used. The material may be any desired parts of the plants but is preferably the above-ground plant parts, e.g. the stalks, leaves, seeds and/or flowers. The material can be used in a very wide range of forms, for example as fresh, still unprocessed material. However, it is advantageous if material is used in dry form. The plant material may be present in comminuted form.

According to one embodiment of the invention, the plant material is subjected to pre-extraction with an aqueous solution before the extraction with the first solvent, and the resulting residue of plant material is further processed. The aqueous solution comprises at least 80%, preferably at least 85%, more preferably at least 90%, even more preferably at least 95%, even more preferably at least 98%, of water. Most preferably, the pre-extraction is carried out using water. This may be tap water or distilled water or water from another source. The temperature of the aqueous solution is preferably 15 to 90° C., more preferably 15 to 80° C., most preferably about 15 to 40° C. The pH of the aqueous solution is not particularly limited and is preferably adjusted to pH 4.5 to 9, more preferably to pH 5.5 to 8, by adding corresponding reagents. The aqueous solution may contain salts or buffer substances. Furthermore, other solvents, such as, for example, polar organic solvents may be present in small amounts. The pre-extraction is as a rule carried out exhaustively. The aqueous pre-extract can be used for dyeing purposes or for pharmaceutical purposes. The aqueous pre-extract can be subjected to drying processes known to the person skilled in the art. The pre-extracted plant material is then extracted with a first solvent.

The first solvent is usually a solution containing 15 to 100% of polar organic solvent in water. Unless stated otherwise, % data in this Application are % by weight. The first solvent can therefore be a substantially pure polar organic solvent without water (100%). However, it may also be a mixture of an aqueous solution and at least one polar organic solvent.

Polar organic solvents in the context of the present Application include alcohols, ketones, carboxylic acids, esters, amides, aldehydes, nitrites, nitro compounds, sulphoxides and organic compounds having a dielectric constant (20° C.) of at least 2.5, preferably at least 5, most preferably at least 10. The polar organic solvent is preferably miscible with water in any ratio.

Polar organic solvents in the context of the present invention are not particularly limited; they are preferably alcohols or ketones. Alcohols having 1 to 4 carbon atoms and ketones having 1 to 4 carbon atoms are particularly preferred. Examples of organic solvents are methanol, ethanol, 1-propanol, isopropanol, 1-butanol, 2-butanol, ethylene glycol, acetone, butanone or any desired mixtures of said solvents. Most preferably, the polar organic solvents are methanol, ethanol, isopropanol or acetone.

The first solvent which is used in step a) is preferably a mixture of an aqueous solution and at least one polar organic solvent. As a rule, the polar organic solvent is present in a proportion of from 15 to 95% in the mixture. A proportion of the polar organic solvent of 20 to 90% is preferred, more preferably from 30 to 80%, most preferably from 40 to 70%. Preferred polar organic solvents in the mixture correspond to the above-mentioned preferred polar organic solvents. A water/alcohol or a water/ketone mixture is particularly preferred.

The extraction can be effected by means of known methods, for example by maceration, percolation or digestion. The extraction can be effected continuously, batchwise, in one stage or in a plurality of stages (Pharmazeutisches Wörterbuch [Pharmaceutical Dictionary], 1993, 7th Edition, de Gryter Verlag, Berlin). At the end, undissolved plant material is usually separated off.

The temperature during the extraction is usually 20 to 95° C., preferably 20 to 80° C., most preferably 30 to 70° C. The temperature during the extraction is most easily established by bringing the extracting agent to the desired temperature prior to the extraction. It is clear to the person skilled in the art that the upper limit of the temperature is determined by the boiling point of the first solvent or of the polar organic solvent in the mixture.

The plant extract thus obtained can be concentrated by removing at least a part of the first solvent. This can be effected in a variety of ways. The term “extract” denotes a composition which is obtainable by an extraction method.

For example, the extract obtained can advantageously be concentrated in vacuo so that at least a part of the polar organic solvent is removed from the extract. A precipitate may form in the receiver after cooling. This can be recovered by filtration, separation or other suitable process techniques. The aqueous phase remaining after the distillation can be worked up completely by concentration and/or drying, even without separating off the precipitate. The drying of the separation precipitate or of the complete aqueous phase can be effected by means of known drying methods, such as, for example, spray drying, fluidized-bed drying, freeze drying or vacuum drying, in order to obtain the product as a powder. The drying can be effected both without and with addition of drying auxiliaries, such as, for example, glucose syrup or maltodextrin. However, the type of drying auxiliary is not particularly limited.

It is also possible to bring about precipitation, for example by cooling, addition of water or acidification of the solution. The precipitate forming is then the product obtained in step b) and can be recovered by customary methods. At least a part of the solvent is removed no later than during recovery of the precipitate.

As a rule, a volume reduction of the extract from step a) takes place in step b). The volume of the extract is preferably reduced to less than 50% of the original volume, more preferably to less than 10%, most preferably to less than 5%. The product obtained in step b) may be a solid, e.g. a powder, a concentrate or a suspension.

After steps a) and b), a flavonoid-enriched composition is obtained.

Surprisingly, it was found that compositions having an even higher luteolin content are obtained if, after the extraction with the first solvent, the extract obtained is concentrated, taken up in a solvent and then precipitated.

According to this embodiment, the product obtained in step b) is taken up in a second solvent and then precipitation of flavonoid-enriched products is brought about so that a solid is obtained. The precipitation can be brought about by acidification or cooling and/or addition of water. These measures can be used individually or in combination.

The second solvent is a solution containing 0 to 100% of polar organic solvents in water. The second solvent may be an aqueous solution without a polar organic solvent (0%). It may also be substantially a pure polar organic solvent (100%). Finally, it is once again also possible to use a mixture of aqueous solution and at least one polar organic solvent. The preferred polar organic solvents which can be used, as were described above for the first solvent, also apply to the second solvent. The product obtained in step b) is, if possible, dissolved in the second solvent. The person skilled in the art is aware that the solubility is influenced by the pH and/or the temperature. Thus, the pH of the second solvent can be changed. It is preferably adjusted to a pH of 5 to 12, preferably 6 to 10, more preferably 7 to 8. The adjustment of the pH can be effected by suitable buffers or alkalis, alkalis being preferred, e.g. sodium hydroxide, ammonia or sodium bicarbonate. The temperature of the second solvent may be higher than room temperature. Thus, the second solvent may be adjusted to a temperature of 30 to 100° C., preferably 40 to 80° C., more preferably 50 to 80° C.

After dissolution of the product in the second solvent—where this is possible—optionally insoluble components can be separated off. Generally known methods, such as filtration, centrifuging, etc., can be used for this purpose. After the dissolution of the product in the second solvent—where this is possible—it is possible to ensure by various measures that a flavonoid-enriched precipitate forms. These measures include in particular acidification of the solution, cooling of the solution and/or addition of water. These measures can be used individually or in combination.

If the solution is acidified after the dissolution, this is preferably effected by adding HCl. The pH thus established is preferably 1 to 6, more preferably 2.5 to 6, most preferably 3 to 5.

If the solution is cooled, the temperature established is less than 20° C., preferably less than 15° C., more preferably less than 10° C. The solution is usually not cooled below 0° C., preferably not below 4° C. The stated upper and lower limits of the temperature can be combined with one another as desired.

If water is added to the solution, it is as a rule distilled water. However, it may also be undistilled water. The water may also be an aqueous solution comprising one or more substances; for example, salts, buffers or other substances may be present. The solution:water volume ratio is as a rule 1:100 to 100:1, preferably 1:10 to 10:1, most preferably 1:3 to 3:1.

If the liquid in which the product obtained in step b) is taken up is an aqueous solution without a polar organic solvent, said solution usually has a pH of 6 to 10. The pH can be adjusted by adding alkalis. Preferably, the aqueous solution has a pH of 7 to 8. The temperature of the aqueous solution is usually 20 to 80° C., preferably 40 to 75° C., more preferably more than 50° C. to 70° C. The aqueous solution is brought into contact with the product obtained in step b), which may be a powder or a concentrate. At least a part of the product dissolves. The components, which may be insoluble under these conditions, can be separated off by filtration, centrifuging, separation, decanting or other customary separation methods. The solution then obtained is adjusted to an acidic pH. The acidic pH is preferably 2.5 to 6.0, more preferably 3.0 to 5.0. The acidic pH is usually established by adding HCl. A precipitate forms as a result. The precipitate preferably forms after cooling of the solution. In this case, the solution can be cooled to less than 20° C., preferably 4 to 15° C., more preferably 4 to 10° C. The resulting precipitate can be recovered in the manner described. This precipitate contains, as a rule, 20 to 30% of luteolin. Unless stated otherwise, data on the luteolin content are based in each case on the dry substance of the extracts (solutions, concentrates or powders).

According to a further embodiment, the product obtained in step b) is taken up with an aqueous solution without a polar organic solvent at temperatures of 30 to 100° C., preferably 50° C. to 90° C., most preferably 60° C. to 80° C. The concentrate is as far as possible dissolved in this heated solution. After cooling of the solution, the resulting precipitate can be recovered and dried in an appropriate manner. Cooling is preferably effected to below 20° C., more preferably to below 15° C., most preferably to below 10° C. According to this embodiment, acidification need not be effected in order to form the precipitate.

In another embodiment, the second solvent contains a polar organic solvent. This is usually a solvent containing 5-100% of a polar organic solvent in water. It was found that a particular increase in the concentration of luteolin can be achieved by a mixture which contains 50 to 100% of polar organic solvent in an aqueous solution. The mixture preferably contains 60 to 100%, most preferably 70 to 100%, of polar organic solvent. The polar organic solvent is preferably an alcohol. The mixture has, as a rule, a temperature of 20 to 80° C., preferably a temperature which corresponds to the boiling point of the polar organic solvent or is up to 10° C. below this. After the product obtained in step b) has been brought into contact with the mixture (second solvent) and, if possible, has been dissolved, water is added to the solution which has been optionally clarified by filtration. A precipitate which contains in particular the aglycones forms. These precipitates can then be worked up as described above. According to this embodiment, compositions having a luteolin content of about 30 to 40% are obtained. If the composition is cooled before the addition of water, a slight precipitate may form and can optionally be separated off. Furthermore, the polar solvent can be partly or completely removed, e.g. by distillation, before the addition of water. The residue is then diluted with water, the precipitate forming.

In a particular embodiment, cooling of the solution and the addition of water can be combined for precipitation. Thus, it is also possible to obtain compositions comprising about 30 to 40% of luteolin.

A further increase in particular in the proportions of aglycones in the composition can be achieved by once again taking up the product obtained in step d) in a mixture of an aqueous solution and at least one polar organic solvent. This dissolution is advantageously effected at temperatures of 30 to 80° C. By adding water to the corresponding solvent extract, it is possible to obtain a precipitate having a further enriched flavonoid content. This precipitate can be recovered and dried in the manner described. This product contains about 35 to 45% of luteolin. The step can be repeated with the product obtained in this manner.

It was also found that a pre-extraction of the plant material with an aqueous solution can be dispensed with when steps c) and d) are carried out.

The invention therefore also relates to a method for the production of a flavonoid-containing composition, which comprises a) extracting material of at least one plant of the Resedaceae family with a first solvent and then separating off insoluble material; b) removing at least a part of the first solvent from the extract obtained in step a); c) taking up the product obtained in step c) in a second solvent and optionally separating off insoluble components; and d) acidifying, cooling and/or adding water to the solution obtained in step c), so that a solid is obtained. For the individual steps, the variants and preferred embodiments described above apply in a corresponding manner.

The invention furthermore relates to flavonoid-containing compositions which are obtainable by a method according to the present invention. The plant extracts and the flavonoid-containing compositions thus obtained can advantageously be used for dyeing textiles and leather and for producing coloured pigments. They can also be used in cosmetics and in medicaments.

By means of the method according to the invention, it is possible to provide flavonoid-containing compositions (plant extracts) having a standardized flavonoid content, in particular luteolin content. The compositions (plant extracts) thus obtained have a high luteolin content of at least 10%, preferably at least 20%, more preferably at least 30%, even more preferably at least 35%, most preferably at least 40%, of luteolin. The invention also relates to such compositions or plant extracts. The extracts preferably contain further flavonoids, such as, for example, apigenin, luteolin-7-glucoside or luteolin-3,7-glucoside.

The flavonoid-containing compositions according to the invention preferably have a flavonoid content of 20 to 90%, based on the total composition. The flavonoid content is more preferably higher than 20%, even more preferably higher than 50%, most preferably higher than 60%.

Furthermore, it was surprisingly found that, by treatment with extracts from the plant Reseda luteola (dyer's rocket), undesired skin conditions can be significantly improved and tautening and smoothing of the skin can be achieved. In particular, a significant improvement in the symptoms of neurodermatitis is achieved by local administration of Reseda extract.

The invention furthermore relates to the use of an extract of at least one plant of the Resedaceae family for treating or preventing undesired skin conditions.

One aspect of the invention is the use of Resedaceae extracts for treating skin diseases. These include neurodermatitis, erythema, burns, Lichen ruber, prurigo, psoriasis, pemphigus, pemphigoid, Dermatitis herpetiformis, sclerodermatitis, Lichen sclerosis, sunburn, Favre Racouchot syndrome, actinic keratoses, Elastosis cutis, common acne, folliculitis simplex and rosacea. The invention also relates to the use of Resedaceae extracts for producing medicaments for treating skin diseases.

A further aspect of the invention is the use of Resedaceae extracts as a cosmetic. Preferred uses of this type are for smoothing the skin, for tautening the skin, for preventing wrinkles, for reducing wrinkles, for reducing the depth of wrinkles, for preventing geroderma, for treating geroderma, for improving the aesthetic impression of the skin, for preventing sunlight-induced skin ageing, for preventing environmentally caused skin changes, for treating environmentally caused skin changes, etc. The invention also relates to the use of Resedaceae extracts for the production of a cosmetic.

Accordingly, the invention relates to the use of an extract from the plant Reseda luteola for improving the tautness of the skin or for retarding or controlling the ageing of the skin. For this purpose, said extract can be incorporated into a cosmetic formulation with a cosmetically tolerated vehicle. The formulation is useful in particular for smoothing the skin, retarding or preventing signs of ageing of the skin, reducing the formation of wrinkles or the depth thereof and alleviating skin irritations. The formulations can be used as antiwrinkle products, as products against ageing of the skin and as skin care products.

The cosmetic formulations of the present invention are suitable for retarding the ageing of the skin and for controlling the ageing of the skin, in particular ageing of the skin which is due to sunlight. Such cosmetic formulations are moreover suitable for treating or preventing skin irritations, such as sunburn and erythemas of the skin.

A particularly suitable use of said cosmetic or pharmaceutical preparations is the use for treating skin diseases. The formulations according to the invention are particularly suitable for treating neurodermatitis.

The present invention also describes methods for the production of Reseda extracts having a particularly high content of flavonoids. In these preparations, the flavonoid content is 10-90%, preferably 40-60%. Extracts according to the invention which have a high flavonoid content are particularly preferred. Suitable extracts for pharmaceutical and/or cosmetic use are not only the extracts produced by the method according to the invention. However, these are particularly preferred.

If the extracts for the pharmaceutical or cosmetic use according to the invention are not produced by the method according to the invention, they may be obtained by methods which are known per se to the person skilled in the art.

The cosmetic or pharmaceutical formulations, in particular the dermatological formulations of the invention, preferably contain 0.001%-10%, preferably 0.01%-10%, more preferably 0.1% 10% and most preferably 1-5% by weight of said extracts of Reseda luteola or other Reseda species, based on the total weight of the formulation.

Said formulations of the invention can advantageously contain further additives, in particular at least one substance which stimulates the synthesis of building blocks of the extracellular matrix of the skin. Examples of such substances are vitamins, in particular vitamins of the A and C group and derivatives thereof, tocopherol, xanthines, in particular caffeine or theophylline, and retinoids, in particular vitamin A acid. Plant extracts, such as extracts of Usnea barbata, Viola tricolor, Calendula officinalis, Rosmarinus officinalis, salvia species, Coriandrum sativum and Iris germanica can also preferably be added. Further additives may consist of sunscreen filters, for example titanium oxides, and vegetable sunscreen filters. The extract of Reseda luteola or other Reseda species, which is used for the treatment according to the invention in formulations for alleviating skin inflammations, in particular neurodermatitis, can advantageously be combined with at least one other active principle which promotes the function of the skin barrier, such as extracts of Potentilla erecta or other tanning agent formulations. Moreover, the Reseda extracts according to the invention can advantageously be used for stabilizing other oxidation-sensitive substances. For example, the combination of hyperforin or a hyperforin-enriched Hypericum extract with Reseda extracts according to the invention is particularly advantageous.

The pharmaceutical or cosmetic composition of the invention may also contain a pharmaceutically or cosmetically tolerated carrier substance. Usually, the carrier substance is suitable for topical applications, does not impair the effect of the active substances and is toxicologically safe. Suitable pharmaceutical carrier substances are described in Remington's Pharmaceutical Sciences, 17th ed., Mack Publishing Company, Easton, Pa. (1990), a standard text book in this area. The concentration of the vehicle in the composition is not particularly limited. It may be 5%-99.99%, preferably 25 to 99.9%, more preferably 50 to 99%, most preferably 50 to 95%, based on the total composition.

The composition may contain further optional substances. CFTA Cosmetic Ingredient Handbook, Second Edition (1992) describes numerous cosmetic and pharmaceutical additives which are used in the industry for skin-care products and medicaments for the skin.

The products according to the invention can be produced with various pharmaceutical formulations. One of the most customary formulations is a topical formulation which is formulated for use on the skin. These usable topical formulations include, without being exclusive, ointments, emulsions, creams, pastes, milk, balsam, gels, lotions, tinctures, plasters, powders, sprays, foams and make-up compositions. Various methods for the production of topical formulations are known per se to the person skilled in the art. They are described in detail in Remington's Pharmaceutical Sciences (see above) or in Pharmaceutical Dosage Forms and Drug Delivery Systems, 6th ed., Williams & Wilkins (1995).

The formulations are preferably applied twice daily by thinly applying the formulation and gently massaging them in. Owing to the good tolerance, the duration of use is not particularly limited.

The use, according to the invention, of Reseda extracts is accordingly based on the application of a cosmetically or pharmaceutically effective proportion of Reseda luteola or other Reseda species, the designated extract being incorporated into a formulation with a cosmetically or pharmaceutically tolerated carrier.

The methods described above permit tolerated and effective alleviation of skin irritations, tautening and smoothing of loose and wrinkled skin, prevention or reduction of light damage and treatment of sunburn and neurodermatitis. The invention relates to a method for treating or preventing undesired conditions of the skin, an effective amount of an extract of at least one plant of the Resedaceae family being administered to an individual, preferably a human. The invention furthermore relates to a method for inhibiting the proliferation of activated mononuclear cells (lymphocytes), a method for inducing apoptosis or preferably in active mononuclear cells (lymphocytes), a method for inhibiting cyclooxygenase, a method for stimulating fibroblasts and/or a method for inhibiting UVA-1-induced toxicity, in each case an effective amount of an extract of at least one plant of the Resedaceae family being administered to an individual. The various embodiments in this Application can be combined with one another. Other claims, properties and advantages of the invention are clearly evident from the examples for the person experienced in the area described.

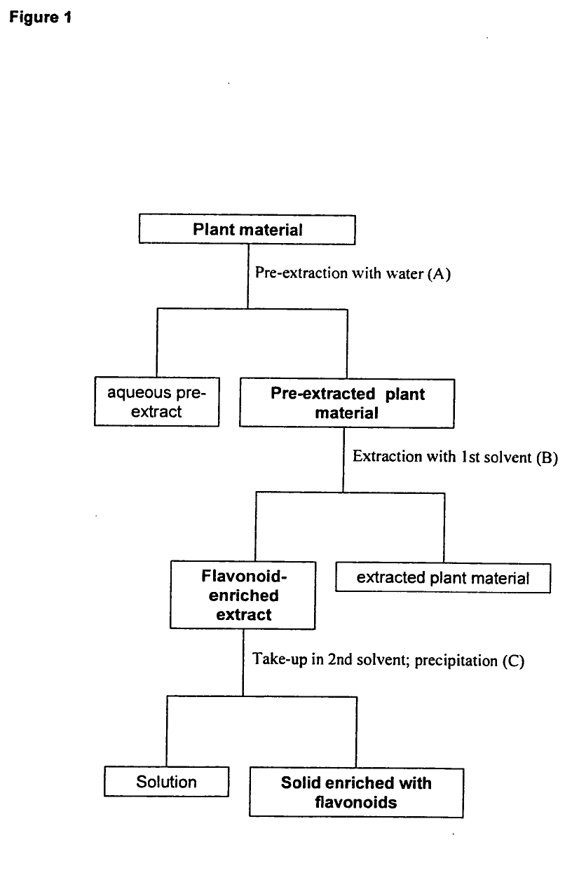

FIG. 1 schematically shows the sequence of the method according to the invention in a certain embodiment. In step A), a pre-extraction of plant material with water takes place. The pre-extracted plant material is subjected to an extraction with a first solvent in step B). The extract obtained is concentrated, and is taken up in a 2nd solvent in step C). After precipitation, a solid enriched with flavonoids is obtained.

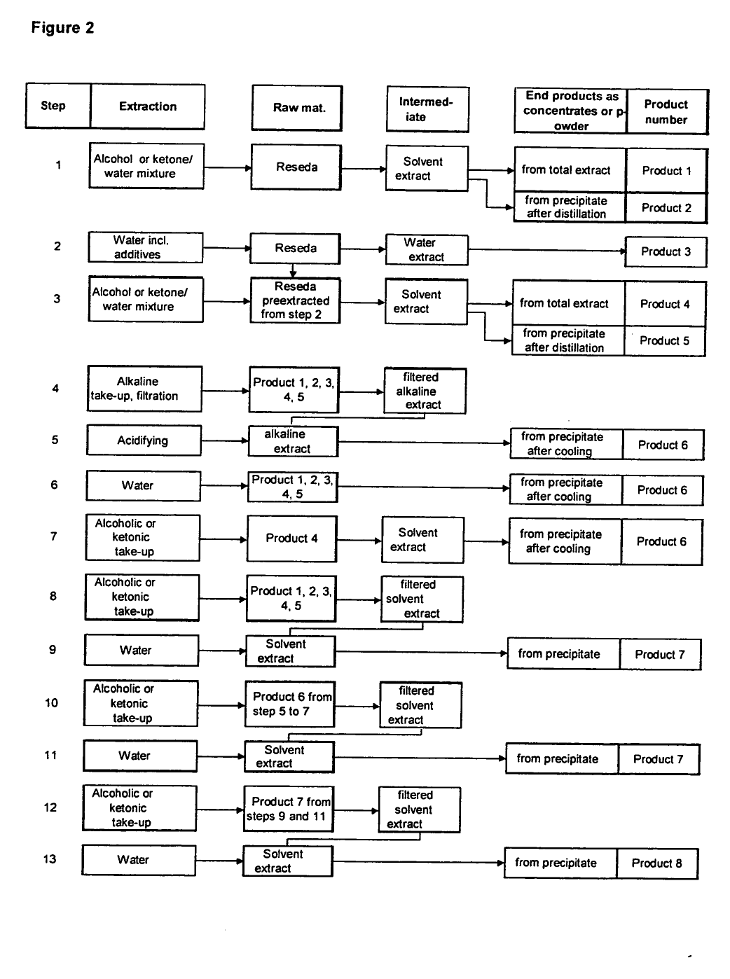

FIG. 2 shows a schematic diagram of different variants of the method for the production of a Reseda extract.

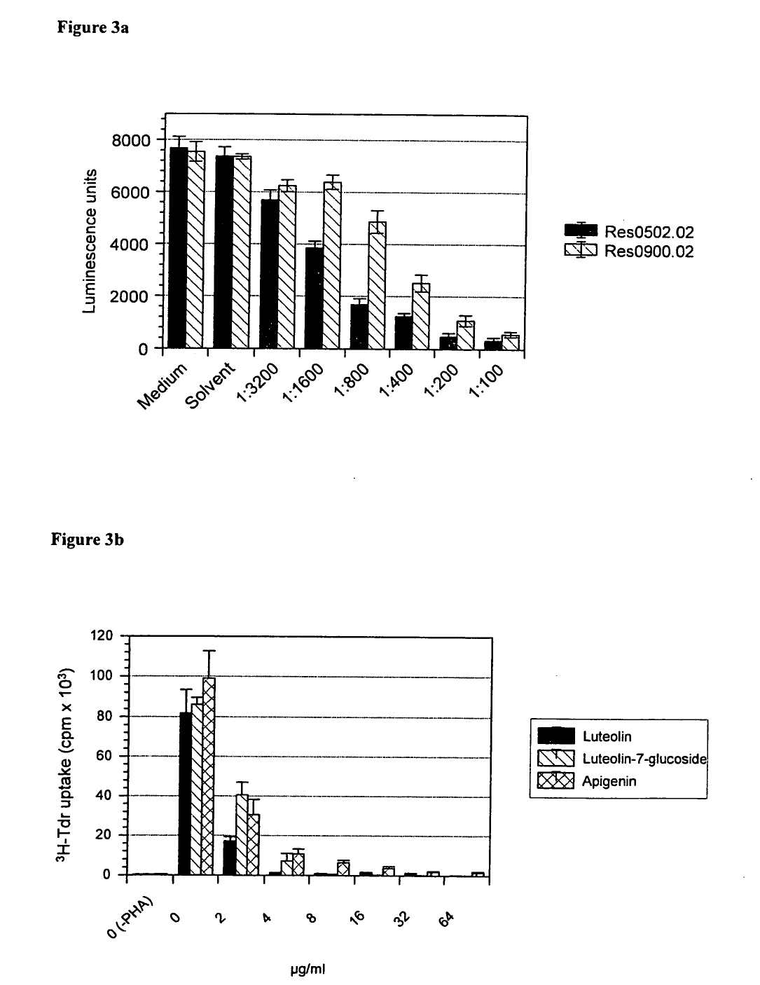

FIG. 3a shows the inhibition of the proliferation of activated mononuclear cells. FIG. 3b shows that luteolin has the strongest proliferation-inhibiting activity (Example 6).

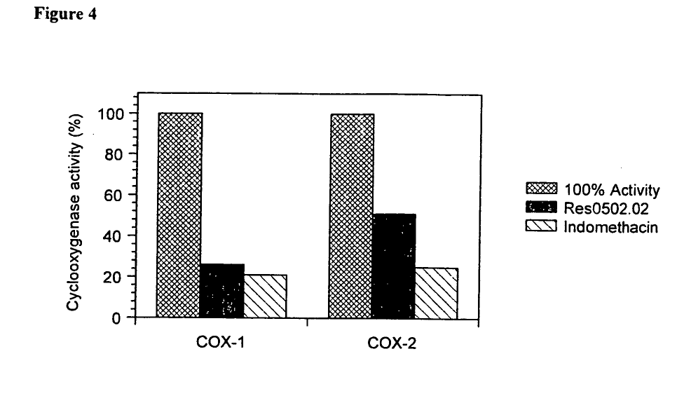

FIG. 4 shows the inhibition of the cyclooxygenases COX-1 and COX-2 in comparison with indomethacin (Example 7).

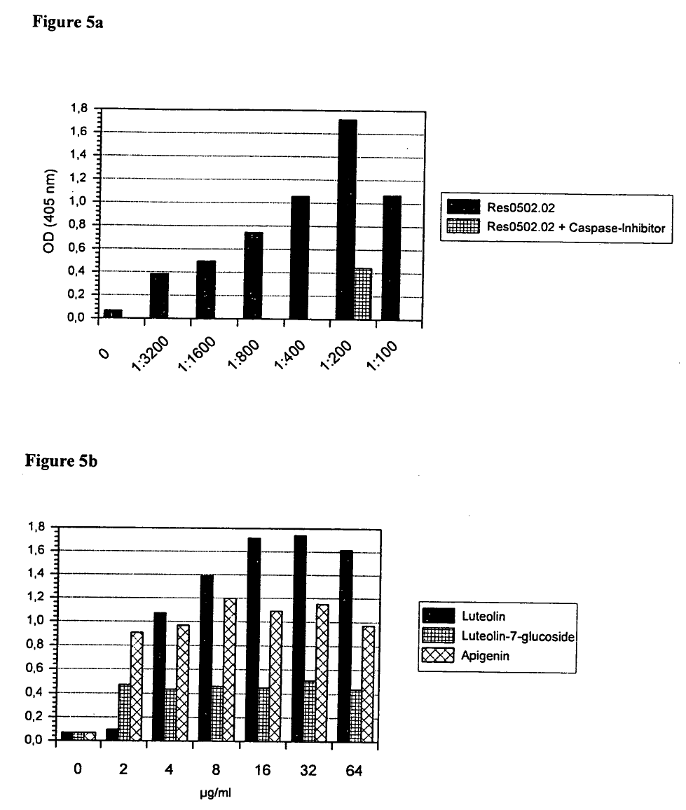

FIG. 5a shows the induction of apoptosis in activated mononuclear cells by Reseda extract. From FIG. 5b, it is clear that luteolin showed a strong apoptosis-inducing effect (Example 12).

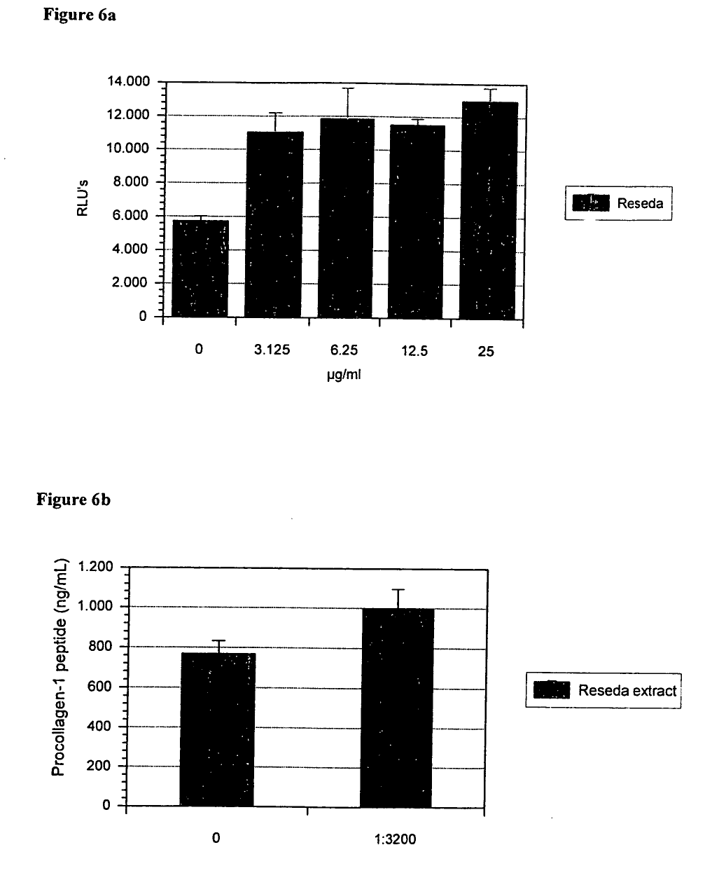

FIG. 6a shows the stimulation of the proliferation of fibroblasts by Reseda extract. FIG. 6b shows that the Reseda extract leads to an increase in collagen synthesis (Example 14).

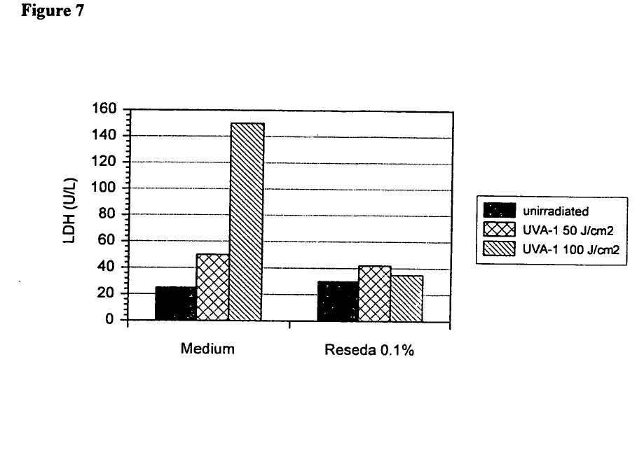

FIG. 7 shows the inhibition of UV-induced toxicity by Reseda extract (Example 15).

The following examples explain the invention in more detail and in no way limit it. The potential applications and the pharmaceutical or cosmetological potential of the Reseda extracts are clearly shown by experimental and clinical examples.

### COMPARATIVE EXAMPLE 1

### Production of a Water Extract

The above-ground plant parts of Reseda are extracted in the dried and comminuted state in the ratio of about 1:5 with water at room temperature. The extract is filtered, concentrated and/or dried. The powder obtained contains about 0.5%-1.5% of luteolin.

### COMPARATIVE EXAMPLE 2

### Production of a Water Extract with Addition of Alkalis

The above-ground plant parts of Reseda are exhaustively extracted in the dried and comminuted state with water with addition of alkalis at a pH of 7-9 at a temperature of 40° C. The extract is filtered, concentrated and/or dried. The powder obtained contains about 2.5%-5% of luteolin.

### COMPARATIVE EXAMPLE 3

### Production of a Solvent Extract of Reseda

The above-ground plant parts of Reseda are exhaustively extracted in the dried and comminuted state with a 50% ethanol/water mixture at a temperature of 50° C. The alcohol is recovered from the filtered extract. The aqueous phase obtained is concentrated and dried. The powder obtained contains about 5%-10% of luteolin.

### EXAMPLE 1

### Production of a Luteolin-Enriched Reseda Extract

The above-ground plant parts of Reseda are extracted in the dried and comminuted state in the ratio of about 1:5 with water at room temperature. The extract is filtered, concentrated and dried. The powder obtained contains about 0.5% to 1.5% of luteolin.

The plant parts extracted with water are exhaustively extracted with a 60% alcohol/water mixture at a temperature of 50° C. The alcohol is recovered from the filtered extract. The aqueous phase obtained is concentrated and dried. The powder obtained contains about 10%-15% of luteolin.

### EXAMPLE 2

### Preparation of a More Highly Luteolin-Enriched Reseda Extract

The powder obtained in Comparative Example 3 is taken up with water in the ratio of 1:5 at the boiling point. After cooling of the solution, the resulting precipitate can be recovered by separation, decanting or filtration.

The precipitate is dried. The powder contains at least 25% of luteolin.

### EXAMPLE 3

### Preparation of a More Highly Luteolin-Enriched Reseda Extract

The powder obtained in Example 1 is taken up with a 50% alcohol/water mixture in the ratio of 1:7 at 60° C. The composition obtained is distilled in order to separate off the alcohol and is diluted 1:1 with water. After cooling, the resulting precipitate can be recovered by separation, decanting or filtration. The precipitate is dried. The powder contains at least 25% of luteolin.

### EXAMPLE 4

### Preparation of a More Highly Luteolin-Enriched Reseda Extract

The products 1, 2, 4 or 5 described (cf. FIG. 2) are taken up with water in the ratio 1:5 at a temperature of 70° C. The pH is adjusted to 7 by adding NaOH. The extract is filtered while hot and is adjusted to a pH of 4 by adding HCl. After cooling of the solution, the resulting precipitate can be recovered by separation, decanting or filtration. The precipitate is dried. The powder contains at least 25% of luteolin.

### EXAMPLE 5

### Preparation of a More Highly Luteolin-Enriched Reseda Extract

The products 1, 2, 4 or 5 described (cf. FIG. 2) are taken up with alcohol or ketone in the ratio of 1:7 at the boiling point. The solution is filtered in the hot state.

The filtrate is cooled. In order to crystallize the flavonoids, water is added to the cooled alcoholic solution in the ratio of 1:3. The mixture is cooled to temperatures of 5-10° C. The resulting precipitate can be recovered in the manner described. The dried precipitate contains at least 30% of luteolin. Further purification is effected by further recrystallization of the precipitate from alcohol with water. The powder obtained in the manner described contains at least 40% of luteolin.

### EXAMPLE 6

### Inhibition of the Proliferation of Stimulated Mononuclear Cells by Reseda luteola Extracts

The test was carried out with the extracts according to the invention as described in Example 1 (Extract Res0900.02) and Example 3 (Extract Res0502.02). Mononuclear cells were obtained from the peripheral blood of a healthy male donor via Ficoll density gradients and cultivated in RPMI medium with 5% foetal calf serum in microtitre plates. The cells were incubated in triplicates with medium (negative control), with solvent (DMSO) or with Reseda extracts dissolved in DMSO, in the concentrations 1:100, 1:200, 1:400, 1:800, 1:1600 and 1:3200. After addition of the additives, the cells were stimulated with 1 μg/ml of phytohaemagglutinin and incubated for 48 h in an incubator. The cell proliferation was then determined from the ATP content of the cells (Via-Light test). A significant, dose-dependent inhibition of cell proliferation by the Reseda luteola extracts was found. The extract produced according to Example 3 was substantially more effective than the extract produced according to Example 1. The solvent had no influence on the proliferation (FIG. 3a).

In the investigation of the individual flavonoids contained in the extracts, it was found that luteolin has the strongest proliferation-inhibiting effect (FIG. 3b). For this purpose, mononuclear cells from the peripheral blood of a health female donor were isolated via Ficoll density gradients and incubated in RPMI medium with 5% foetal calf serum in microtitre plates. The cells were incubated in triplicates with the flavonoids luteolin, luteolin-7-glucoside and apigenin. The pure substances were dissolved in 70% ethanol and further diluted in medium. The final concentrations were 64, 32, 16, 8, 4, 2 and 0 μg/ml. After addition of the flavonoids, the cells were stimulated with 1 μg/ml of phytohaemagglutinin and incubated for 24 h in an incubator at 37° C. After addition of 1 μCurie of radioactive ³H-thymidine, the cells were incubated for a further 18 h, and the incorporated radioactivity was then measured in a scintillation counter (Canberra-Packard).

### EXAMPLE 7

### Inhibition of Cyclooxygenase-1 (COX-1) and COX-2 by the Extract According to the Invention as Described in Example 3 (Res0502.02) in Comparison with the Nonsteroidal Antiphlogistic Agent Indomethacin

A cell-free enzyme inhibition kit from Cayman Chemicals (product number #760111) serves as a detection method. The assay is carried out at 25° C. in order to ensure optimum enzyme function. Recombinant COX-1 or COX-2 is added to a defined heme-containing assay buffer. Thereafter, Reseda extract Res0502.02 according to Example 3 in a final concentration of 1:200 or indomethacin (final concentration 10 μM) and a colour substrate are added. After addition of the enzyme substrate arachidonic acid, the reaction takes place in five minutes, and the reaction of the colour substrate, which is coupled to the formation of the endoperoxide PGH2, is measured calorimetrically. The untreated enzyme reaction is defined as 100% activity and the inhibition achieved is expressed in relation thereto. It is found that the Reseda extract produces inhibition of COX-1 and, somewhat more weakly, of COX-2 which is comparable with indomethacin (FIG. 4).

### EXAMPLE 8

### Healing Effect of Reseda luteola Cream in the Treatment of Neurodermatitis

a) A 31 year old man with neurodermatitis of the lower arm which had existed for several weeks was treated with the Reseda luteola cream mentioned under Example 9 for 10 days. This resulted in virtually complete healing of the skin lesions. Moreover, the depth of wrinkles decreased and the tautness of the skin increased.

b) A 22 year old man with neurodermatitis of the elbow was treated for one week with the Reseda luteola cream described under Example 9. In the follow-up examination, the redness was found to have almost completely disappeared.

c) A 24 year old woman with therapy-resistant neurodermatitis which had persisted for many years was treated with the Reseda luteola cream described under Example 9, in comparison with the strongly anti-inflammatory Protopic® ointment 0.1%, for two weeks. The Reseda luteola cream was applied to the right wrist, and the Protopic® cream to the left wrist. In the follow-up examination, an improvement of the symptom, which corresponded with the side treated with Protopic®, was found on the skin treated with Reseda luteola. On the side treated with Reseda luteola, a substantial reduction in the depth of wrinkles and tautening of the skin were additionally found.

d) A 68 year old woman suffered from eczema of the face with dryness, peeling and painful fissures. After treatment for 10 days with Reseda luteola cream according to Example 9, healing of the fissures and a substantial decline of erythema and peeling had occurred.

### EXAMPLE 9

### Production of a Reseda Luteola Cream for Intensive Care of Inflamed Skin

| | |
|---|---|
| Extract of Reseda luteola according to | 2.0 |
| Example 3 | |
| Glyceryl monostearate 60 | 4.0 |
| Cetyl alcohol | 6.0 |
| Medium-chain triglycerides | 7.5 |
| Squalane | 25.5 |
| Macrogol-20-glyceryl monostearate | 7.0 |
| Propylene glycol | 10.0 |
| Purified water | to 100 |

The components in addition to the Reseda extract form the cream base. The components of the cream base may be present in ratios other than those stated. The cream base may comprise other and/or further substances.

### EXAMPLE 10

### Cosmetic Care Gel for the Treatment of Wrinkles and Skin Irritations on the Face

| | |
|---|---|
| Aqueous extract of Reseda luteola according to | 1.0 g |
| Example 3 | |
| Extract of Potentilla erecta | 0.5 g |
| Carbomer 50,000 | 0.5 g |
| 2-Propanol | 5.0 g |
| Propylene glycol | 10.0 g |
| Sodium hydroxide | 0.12 g |
| Purified water | to 100 |

The components in addition to the plant extracts form the gel base. The gel base may also contain the components in other ratios. Other and/or further substances may be present.

### EXAMPLE 11

### Special Ointment for the Treatment of Dry Skin Conditions

| | |
|---|---|
| Extract of Reseda luteola according to | 1.0 g |
| Example 3 | |
| Calendula officinalis CO2 extract | 3.0 g |
| Sodium dodecylsulphate | 5.0 g |
| Medium-chain triglycerides | 5.0 g |
| Glyceryl monostearate 60 | 20.0 g |
| Carnauba wax | 20.0 g |
| Squalane | 25.0 g |
| 2-Octyldodecanol | 25.0 g |

The components in addition to the plant extracts form the ointment base. The ointment base may also contain the components in other ratios. Other and/or further substances may be present.

### EXAMPLE 12

### Induction of Apoptosis in Activated Mononuclear Cells (Lymphocytes)

Mononuclear cells (lymphocytes) were incubated with solvent, with medium and with luteolin-enriched Reseda extract according to Example 3 in the concentrations 1:1000 (100 μg/ml), 1:200 (50 μg/ml), 1:400 (25 μg/ml), 1:800 (12.5 μg/ml), 1:1600 (6.25 μg/ml) and 1:3200 (3.125 μg/ml). “0” in FIG. 5a means that no Reseda extract was present. After addition of the additives, the cells were stimulated with 1 μg/ml of phytohaemagglutinin and incubated for 48 h in an incubator. The apoptosis induction was then determined by measuring the oligonucleosomes (low molecular weight DNA fragments) formed in the cells (cell death detection ELISA). The extracts were found to cause a significant, dose-dependent increase in the apoptosis rate. The apoptosis induction could be blocked by a caspase inhibitor, i.e. is caspase-induced (FIG. 5a). In the investigation of the individual flavonoids contained in the extracts, it was found that luteolin has the strongest apoptosis-inducing activity (FIG. 5b).

### EXAMPLE 13

### Antioxidant Effect of Reseda Extract

The relative antioxidant effect of Reseda extract was determined by the TEAC III method in comparison with Trolox and ascorbic acid:

| | |
|---|---|
| Trolox: | 1.0 |
| Ascorbic acid | 1.1 |
| Reseda extract | 1.5 |

The TEAC III method and other methods are described in Böhm V. (2000) Ernährungsumschau 47, pages 372-375.

### EXAMPLE 14

### Stimulation of Fibroblasts

In a further test, the activity of a luteolin-enriched Reseda extract was investigated with regard to the proliferation of primary human fibroblasts. It was surprisingly found that the extract, in very low concentrations, stimulates the proliferation of the fibroblasts (ViaLight assay) (FIG. 6a) and leads to an increase in collagen synthesis (procollagen 1 C peptide EIA) (FIG. 6b). The Reseda extract No. 3=Res0502.02 was used. The concentrations 25 μg/ml, 12.5 μg/ml, 6.25 μg/ml and 3.125 μg/ml correspond to the dilutions 1:400, 1:800, 1:1600 and 1:3200, respectively. While the concentrations are plotted in FIG. 6a, the dilution of the Reseda extract is shown in FIG. 6b. “0” in FIG. 6b means that no Reseda extract was present.

### EXAMPLE 15

### Inhibition of UV-Induced Toxicity by Reseda Extract

Model: LDH Liberation from Primary Fibroblasts

Subconfluent primary human fibroblasts were exposed to 0/50/100 J/cm² or UVA-1 in PBS buffer. The cells were then incubated in cell culture medium with and without addition of Reseda extract (0.1%) for 24 h. The liberation of lactate dehydrogenates (LDH) was then measured in the supernatant (Mira-Cobas enzyme test). The addition of Reseda extract in low concentration virtually completely inhibits the UVA-1-induced damage to the cell membrane. Reseda extract according to Example 3 was used (Res0502.02). The concentration 0.1% corresponds to a dilution of 1:1000.

1. A method for the production of a flavonoid-containing composition, which comprises
a) extracting material of at least one plant of the Resedaceae family, which has been pre-extracted with an aqueous solution, with a first solvent and
b) removing at least a part of the first solvent from the extract obtained in step a).

2. The method according to claim 1, characterized in that material of the plant variety Reseda luteola L. is used.

3. The method according to claim 1, characterized in that the first solvent is a water/alcohol or a water/ketone mixture.

4. The method according to claim 1, characterized in that a distillation of the extract obtained in step a) is carried out in step b).

5. The method according to claim 1, characterized in that it furthermore comprises
c) taking up the product obtained in step b) in a second solvent and optionally separating off insoluble components; and
d) acidifying, cooling and/or adding water to the solution obtained in step c) so that a solid is obtained.

6. The method according to claim 5, characterized in that the product obtained in step b) is taken up in an aqueous solution having a pH of 6 to 10, the insoluble components are separated off, the solution thus obtained is adjusted to a pH of 2.5 to 6 and the resulting solid is recovered.

7. The method according to claim 5, characterized in that the product obtained in step b) is taken up in water having a temperature of 30 to 100° C., the resulting solution is cooled and the resulting solid is recovered.

8. The method according to claim 5, characterized in that the product obtained in step b) is taken up in a water/alcohol or a water-ketone mixture having a temperature of 20 to 80° C., water is added and the resulting solid is recovered.

9. The method according to claim 5, characterized in that the product obtained in step b) is taken up in a water/alcohol or water/ketone mixture, insoluble components are removed, water is then added and the resulting solid is recovered.

10. The method according to claim 5, characterized in that the solid obtained in step d) is taken up with a water/alcohol or a water/ketone mixture, insoluble components are removed, water is added and the resulting solid is recovered.

11. The method according to claim 10, characterized in that the solid obtained is again taken up with a water/alcohol or a water/ketone mixture, insoluble components are removed, water is added and the resulting solid is recovered.

12. A method for the production of a flavonoid-containing composition, which comprises
a) extracting material of at least one family of the Resedaceae family with a first solvent;
b) removing at least a part of the first solvent from the extract obtained in step a);
c) taking up the product obtained in step b) in a second solvent and optionally separating off insoluble components; and
d) acidifying, cooling and/or adding water to the solution obtained in step c) so that a solid is obtained.

13. The method according to claim 12, characterized in that the solid obtained in step d) is taken up with a water/alcohol or a water/ketone mixture, insoluble components are removed, water is added and the resulting solid is recovered.

14. The method according to claim 13, characterized in that the solid obtained is taken up with a water/alcohol or a water/ketone mixture, insoluble components are removed, water is added and the resulting solid is recovered.

15. A flavonoid-containing composition obtainable by a method according to claim 1.

16. The flavonoid-containing plant extract comprising at least 10% of luteolin.

17. A method for dyeing textiles or leather or for producing pigments comprising the use of a flavonoid-containing composition according to claim 15.

18. A method of treating or preventing undesired conditions of the skin comprising the step of administering to a subject in need thereof a medicament comprising a composition according to claim 15 or an extract of at least one plant of the Resedaceae family.

19. The method according to claim 18, characterized in that the undesired condition of the skin is selected from the group consisting of neurodermatitis, Lichen ruber, prurigo, psoriasis, pemphigus, pemphigoid, Dermatitis herpetiformis, sclerodermatitis, Lichen sclerosis, sunburn, Favre Racouchot syndrome, actinic keratoses, Elastosis cutis, common acne, Folliculitis simple and rosacea.

20. The method according to claim 18, characterized in that the undesired condition of the skin is sunburn.

21. The method according to claim 18, characterized in that plant extract is capable of tautening and/or smoothing the skin.

22. The method according to claim 18, characterized in that the plant extract is capable of preventing wrinkles and/or light-induced ageing of the skin.

23. The method according to claim 18, wherein said medicament comprises a topical formulation is selected from the group consisting of ointments, emulsions, creams, pastes, milk, balsam, gels, lotions, tinctures, plasters, powders, sprays, foams and make-up compositions.

24. A skin-care product comprising a composition according to claim 15 or an extract of at least one plant of the Resedaceae family.

25. The skin-care product according to claim 24, characterized in that it furthermore contains a further plant extract which does not originate from a plant of the Resedaceae family.

26. The skin-care product according to claim 24, characterized in that it is selected from the group consisting of ointments, emulsions, creams, pastes, milk, balsam, gels, lotions, tinctures, plasters, powders, sprays, foams and make-up compositions.
